(19) United States
(12) Patent Application Publication
(10) Pub. No.: US 2019/0287662 A1
(43) Pub. Date: Sep. 19, 2019

(54) SYSTEM AND METHOD FOR GENERATING A COMPREHENSIVE INDIVIDUALIZED ELECTRONIC HEALTH INFORMATION MONETIZATION PLATFORM

(71) Applicant: Taproot Research Inc., Washington, DC (US)

(72) Inventors: Alicia HENNIE, Washington, DC (US); Jeffrey HENNIE, Washington, DC (US); Patrick NEILER, Canyon Country, CA (US); David SANDERS, Alexandria, VA (US); Brian AHIER, Front Royal, VA (US)

(21) Appl. No.: 16/354,955

(22) Filed: Mar. 15, 2019

Related U.S. Application Data
(60) Provisional application No. 62/643,983, filed on Mar. 16, 2018.

(51) Int. Cl. G16H 10/60; G06F 21/62; G06F 21/31

(52) U.S. Cl. CPC G16H 10/60; G06F 21/6245; G06F 2221/2115; G06F 21/6272; G06F 21/31

(57) ABSTRACT

A system for generating a comprehensive individualized electronic health information monetization platform, which will facilitate the management, monitoring, and monetization of an individual's personal health information (PHI). The system generates comprehensive individualized electronic health information monetization platform, and includes a data storage containing user identification information, user electronic health information, user financial information, and one or more user acquisition preferences, a data source generator, and an electronic health information transaction processor. An electronic health information transaction processor may upload the de-identified synthesized data from the data source generator, parse the de-identified synthesized data to build a search index, evaluate the data to generate a data acquisition trending model, generate a comprehensive individualized electronic health information monetization platform utilizing the data acquisition trending model that determines and recommends data samples that are compatible.

### CROSS-REFERENCE TO RELATED APPLICATIONS

This application claims priority from U.S. Provisional Application No. 62/643,983, filed on Mar. 16, 2018, the disclosure of which is incorporated herein by reference in its entirety.

### FIELD OF THE DISCLOSURE

The present disclosure relates to systems and methods for generating a comprehensive individualized electronic health information monetization platform. The system will facilitate the generation and housing of a complete record of an individual's health data assets within an application, which may be available to third parties for purchase (lease).

### BACKGROUND OF THE DISCLOSURE

Currently, individual data owners have no way to participate in the health IT marketplace. Individuals are excluded from the monetization of their health information assets including information from genomic and medical records.

Additionally, health big data research lags behind other sectors because to access an individual's complete health information history requires multiple data requests and agreements with the various health providers as interoperability has not yet reached the health sector. Patients also have information about lifestyle and life choices that is not recorded.

These and other drawbacks exist.

### SUMMARY OF THE DISCLOSURE

Various embodiments of the present disclosure provide systems and methods for a comprehensive individualized electronic health information monetization platform, which will facilitate the management, monitoring, and monetization of an individual's personal health information (PHI). The connected data may include, for example, data from electronic health records (EHRs) or electronic medical records (EMRs), genome sequences (DNA), data from health and fitness trackers, including wearable devices, and Medicare and Medicaid data. For example, the system may interface with APIs relative to the users health providers, DNA providers and fitness devices. The data may be transferred securely to data storage following regulatory standards, and the system may dynamically provide, and maintain, in real time, a Health Insurance Portability and Accountability Act (HIPAA) compliant platform. Additionally, EHR data that is imported may pass through standardization layer and be formatted to adhere to the Fast Healthcare Interoperability Resources (FHIR) specifications, which may normalize all EHR data, which will be essential for the discovery and usefulness of the data in medical research studies.

The system may collect, protect, and aggregate personal health information assets for auction to third party purchases, that may include researchers working to cure, treat and prevent disease, and the platform may provide researchers with access to the most complete health information database. For example, sellers may opt-in to authorize the system to collect their health information in one place. Buyers may purchase data sets curated from the sellers' accounts. The associated data may be anonymized, per agreement with sellers during signup, and non-anonymized under special agreement with sellers for specific use cases. Researchers may query the system data storage by establishing a search criteria against EHR resources. The system will return only patient records that match the criteria, and will be available to purchase alongside the DNA and Trackers, if desired.

A system for generating a comprehensive individualized electronic health information monetization platform may include a data storage containing user identification information, user electronic health information, user financial information, and one or more user acquisition preferences, a data source generator, and an electronic health information transaction processor. The data source generator may take as inputs user electronic health information from third party electronic health information systems, and cluster the received user identification information, user electronic health information, user financial information, and one or more user acquisition preferences. The data source generator may utilize a data cleanser that utilizes bioinformatics, artificial intelligence and machine learning technologies to clean and synthesize the received user electronic health information based on data type and structure. The data source generator may de-identify the synthesized data, and encrypt the de-identified synthesized data for transmission. An electronic health information transaction processor may upload the de-identified synthesized data from the data source generator, parse the de-identified synthesized data to build a search index, evaluate the data to generate a data acquisition trending model, generate a comprehensive individualized electronic health information monetization platform utilizing the data acquisition trending model that determines and recommends data samples that are compatible, and transmit via a communication interface, a push notification to a user device. The push notification may include a data acquisition request received from transaction server. The data storage may store the generated data acquisition trending model.

In one embodiment authentication processor may be connected to the electronic health information transaction processor that receives user data and user input associated with an authentication request, sent from the user device via the communication interface, to authenticate the user. Upon authentication of the user based on evaluation of the user data and user input, the system may transmit a secure link to the user device that provides access to the comprehensive individualized electronic health information monetization platform. The authentication processor may confirm the location of the user device over a wireless connection by evaluating a unique user ID-secure link token pair. The authentication processor may confirm the authenticity of the data source of the user electronic health information from third party electronic health information systems by evaluating a unique user ID-secure link token pair.

The electronic health information transaction processor may receive recommended data acquisition requests and may automatically update the comprehensive individualized electronic health information monetization platform with the received recommended data acquisition requests.

The electronic health information transaction processor may establish a secure communication interface with a third party system configured to share a secure link that provides access to the comprehensive individualized electronic health information monetization platform. The electronic health information transaction processor may utilize the data acquisition trending model to generate a user's data net worth based on a system generated data density score associated with the user's electronic health information. The electronic health information transaction processor may transmit via a communication interface, a push notification to the user device. The push notification may include a secure link to input additional user to increases the user's generated data net worth.

Upon receipt of acceptance of the data acquisition request by the user device, the transaction server may process an associated transaction associated with a data acquisition request received from transaction server. The electronic health information application processor may link the de-identified synthesized data with the user's account via a unique identifier.

The system utilizes and interoperable framework, and machine learning and natural language processing technologies that provide and facilitate data acquisition requests. A persistent secure connection across distributed systems facilitates automatic synchronization of the data items across the distributed systems and devices accessing those systems.

Accordingly, they system may consolidate and synchronize and individual's personal health data in real time to provide the centralized patient health data necessary to generate the monetization platform. Utilizing a persistent bi-directional connection, the online registry electronic health information transaction application process processor may automatically synchronize the health information monetization platform in real time as data acquisition requests are accepted, declined, and associated financial transactions are executed.

A system application may be utilized for sellers to have the option to turn down offers to lease their data to foster competition. Buyers may offer different prices for data of different values to foster asset management by the sellers. High value data could be more complete, for example, it may display genetic markers for in demand diseases.

### BRIEF DESCRIPTION OF THE DRAWINGS

Various embodiments of the present disclosure, together with further objects and advantages, may best be understood by reference to the following description taken in conjunction with the accompanying drawings, in the several Figures of which like reference numerals identify like elements, and in which:

FIG. 1 depicts an example system for generating a comprehensive individualized electronic health information monetization platform, according to embodiments of the disclosure;

FIG. 2 depicts an example system, according to embodiments of the disclosure;

FIG. 3 depicts an example communication architecture that may be used to implement one or more embodiments of the disclosure;

FIG. 4 depicts an example data access architecture that may be used to implement one or more embodiments of the disclosure;

FIG. 5 is a diagram illustrating a comprehensive individualized health information monetization platform, according to embodiments of the disclosure;

FIG. 6 is a diagram illustrating a comprehensive individualized health information monetization platform, according to embodiments of the disclosure;

FIG. 7 is an example flowchart for a method for generating a comprehensive individualized electronic health information platform, according to embodiments of the disclosure; and

FIGS. 8-12 depict example customizable interfaces, according to embodiments of the disclosure.

### DETAILED DESCRIPTION OF THE EMBODIMENTS

The following description is intended to convey a thorough understanding of the embodiments described by providing a number of specific example embodiments and details involving generating a comprehensive individualized electronic health information monetization platform. It should be appreciated, however, that the present disclosure is not limited to these specific embodiments and details, which are examples only. It is further understood that one possessing ordinary skill in the art, in light of known systems and methods, would appreciate the use of the invention for its intended purposes and benefits in any number of alternative embodiments, depending on specific design and other needs. An electronic health record (EHR) system and client device are used as examples for the disclosure. The disclosure is not intended to be limited to EHR systems and client devices only. For example, many other electronic systems and devices may generate a comprehensive individualized data record monetization platform.

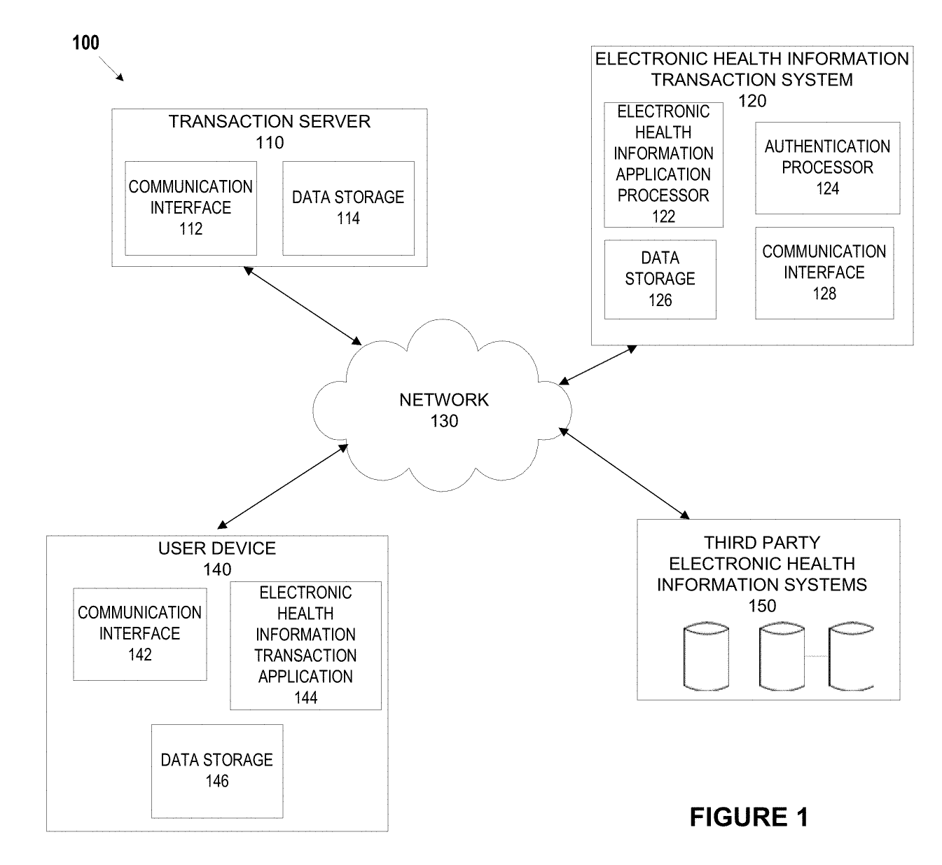

FIG. 1 depicts an example system 100 for generating a comprehensive individualized electronic health information monetization platform. As shown in FIG. 1, an example system 100 may include one or more transaction servers 110, one or more electronic health information transaction systems 120, one or more user devices, and one or more third party electronic health information systems 150 connected over one or more networks 130.

For example, network 130 may be one or more of a wireless network, a wired network or any combination of wireless network and wired network. For example, network 130 may include one or more of a fiber optics network, a passive optical network, a cable network, an Internet network, a satellite network, a wireless LAN, a Global System for Mobile Communication (“GSM”), a Personal Communication Service (“PCS”), a Personal Area Network (“PAN”), Wireless Application Protocol (WAP), Multimedia Messaging Service (MMS), Enhanced Messaging Service (EMS), Short Message Service (SMS), Time Division Multiplexing (TDM) based systems, Code Division Multiple Access (CDMA) based systems, D-AMPS, Wi-Fi, Fixed Wireless Data, IEEE 802.11b, 802.15.1, 802.11n and 802.11g, a Bluetooth network, or any other wired or wireless network for transmitting and receiving a data signal.

In addition, network 130 may include, without limitation, telephone lines, fiber optics, IEEE Ethernet 902.3, a wide area network (“WAN”), a local area network (“LAN”), a wireless personal area network (“WPAN”), or a global network such as the Internet. Also network 130 may support an Internet network, a wireless communication network, a cellular network, or the like, or any combination thereof. Network 130 may further include one network, or any number of the example types of networks mentioned above, operating as a stand-alone network or in cooperation with each other. Network 130 may utilize one or more protocols of one or more network elements to which they are communicatively coupled. Network 130 may translate to or from other protocols to one or more protocols of network devices. Although network 130 is depicted as a single network, it should be appreciated that according to one or more embodiments, network 130 may comprise a plurality of interconnected networks, such as, for example, the Internet, a service provider's network, a cable television network, corporate networks, and home networks.

User device 140 may include, for example, one or more mobile devices, such as, for example, personal digital assistants (PDA), tablet computers and/or electronic readers (e.g., iPad, Kindle Fire, Playbook, Touchpad, etc.), wearable devices (e.g., Google Glass), telephony devices, smartphones, cameras, music playing devices (e.g., iPod, etc.), televisions, set-top-box devices, and the like.

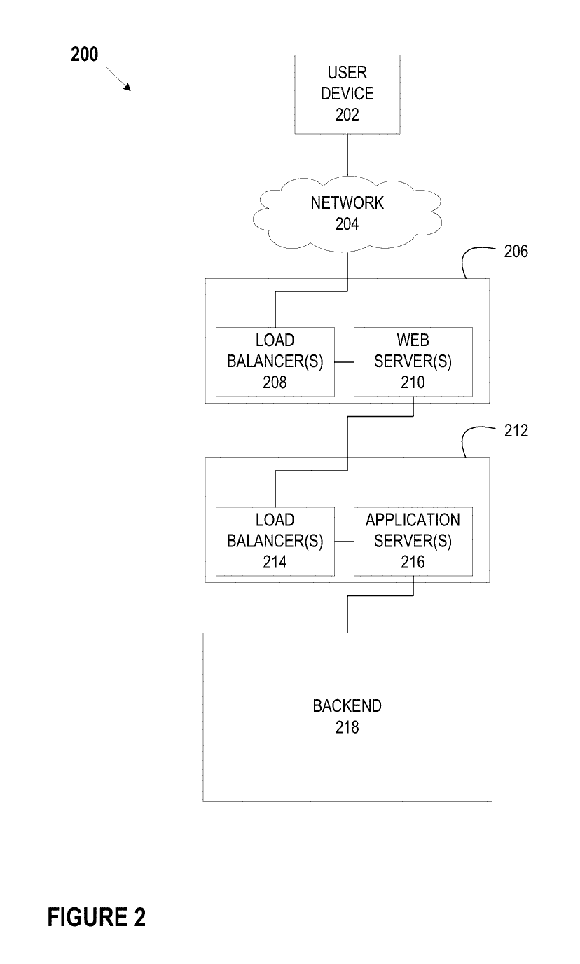

Transaction server 110, electronic health information transaction system 120, user device 140, and third party electronic health information systems 150 also may include a network-enabled computer system and/or device. As referred to herein, a network-enabled computer system and/or device may include, but is not limited to: e.g., any computer device, or communications device including, e.g., a server, a network appliance, a personal computer (PC), a workstation, a mobile device, a phone, a handheld PC, a personal digital assistant (PDA), a thin client, a fat client, an Internet browser, or other device. The network-enabled computer systems may execute one or more software applications to, for example, receive data as input from an entity accessing the network-enabled computer system, process received data, transmit data over a network, and receive data over a network. For example, account provider system may include components such as those illustrated in FIG. 2.

Transaction server 110, electronic health information transaction system 120, user device 140, and third party electronic health information systems 150, may include at least one central processing unit (CPU), which may be configured to execute computer program instructions to perform various processes and methods. Transaction server 110, electronic health information transaction system 120, user device 140, and third party electronic health information systems 150 may include data storage, including for example, random access memory (RAM) and read only memory (ROM), which may be configured to access and store data and information and computer program instructions. Data storage may also include storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, any type of tangible and non-transitory storage medium), where the files that comprise an operating system, application programs including, for example, web browser application, email application and/or other applications, and data files may be stored. The data storage of the network-enabled computer systems may include electronic information, files, and documents stored in various ways, including, for example, a flat file, indexed file, hierarchical database, relational database, such as a database created and maintained with software from, for example, Oracle® Corporation, Microsoft® Excel file, Microsoft® Access file, a solid state storage device, which may include an all flash array, a hybrid array, or a server-side product, enterprise storage, which may include online or cloud storage, or any other storage mechanism.

Transaction server 110, electronic health information transaction system 120, user device 140, and third party electronic health information systems 150 may further include, for example, a processor, which may be several processors, a single processor, or a single device having multiple processors. Although depicted as single elements, it should be appreciated that according to one or more embodiments, transaction server 110, electronic health information transaction system 120, user device 140, and/or third party electronic health information systems 150 may comprise a plurality of transaction servers 110, electronic health information transaction systems 120, user devices 140, and third party electronic health information systems 150.

As shown in FIG. 1, each transaction server 110, electronic health information transaction system 120, user device 140, and third party electronic health information systems 150 may include various components. As used herein, the term “component” may be understood to refer to computer executable software, firmware, hardware, and/or various combinations thereof. It is noted there where a component is a software and/or firmware component, the component is configured to affect the hardware elements of an associated system. It is further noted that the components shown and described herein are intended as examples. The components may be combined, integrated, separated, or duplicated to support various applications. Also, a function described herein as being performed at a particular component may be performed at one or more other components and by one or more other devices instead of or in addition to the function performed at the particular component. Further, the components may be implemented across multiple devices or other components local or remote to one another. Additionally, the components may be moved from one device and added to another device, or may be included in both devices.

As depicted in FIG. 1, a transaction server 110 may include a communication interface 112 and data storage 114. Transaction server 110 may be configured to provide real-time user data acquisition determinations, and may include various hardware and software components to communicate, via communication interface 112, between a data acquisition system, account provider system and/or a user device to process a transaction such as a purchase of user data. In one embodiment, transaction server 110 may be utilized by a customer user, for example, a researcher to search for data meeting specific system specified data, make an offer to purchase the data, and facilitate the purchase transaction by an associated account provider system.

The transaction server 110 may be part of the backend computing systems and associated servers of a data acquisition system, account provider systems, and the like. An account provider system may include, by way of example and not limitation, depository institutions (e.g., banks, credit unions, building societies, trust companies, mortgage loan companies, pre-paid gift cards or credit cards, etc.), contractual institutions (e.g., insurance companies, pension funds, mutual funds, etc.), investment institutions (e.g., investment banks, underwriters, brokerage funds, etc.), and other non-bank financial institutions (e.g., pawn shops or brokers, cashier's check issuers, insurance firms, check-cashing locations, payday lending, currency exchanges, microloan organizations, crowd-funding or crowd-sourcing entities, third-party payment processors, etc.).

Data storage 114 may store data and/or components to enable the generation, transmission and processing of user data associated with an account (e.g., card number, account type, account balance, account limits, budget data, recent transactions, pairing data such as time and date of pairing with a mobile device, and the like) and account holder data (e.g., account holder name, address, phone number(s), email address, demographic data, and the like).

Transaction server 110 may include components to send and/or receive data for use in other components, such as communication interface 112. A communication interface 112 may include various hardware and software components, such as, for example, a repeater, a microwave antenna, a cellular tower, or another network access device capable of providing connectivity between network mediums. The communication interface 112 may also contain various software and/or hardware components to enable communication over a network 130. For example, communication interface 112 may be capable of sending or receiving signals via network 130. Moreover, communication interface 112 may provide connectivity to one or more wired networks and may be capable of receiving signals on one medium such as a wired network and transmitting the received signals on a second medium such as a wireless network.

Electronic health information transaction system 120 may include electronic health information transaction application processor 122, authentication processor 124, data storage 126, and communication interface 128. Electronic health information transaction system 120 may include data and/or components, systems, and interfaces, including application programming interfaces (APIs) to enable the generation, transmission, and processing of data including digital authentication data. Electronic health information transaction application processor 122 may receive from transaction server 110 a data purchase request approval including at least customer identification information, customer financial information, and one or more customer data sale preferences that may be retrieved from data storage 126. Utilizing machine learning and natural language processing technologies, electronic health information transaction processor 122 may cluster and evaluate information retrieved from third party electronic health information systems 150, to generate a data acquisition trending model, which may be stored in data storage 126, to determining and recommending data samples that are compatible with the data acquisition. The system may also generate a user's data net worth, as well a data density score associated with a user's particular data processed by the system. Electronic health information transaction application processor 122 may build content to be processed by the machine learning and natural language processing technologies and learning models based on factors associated with the acquisition, which may include location of the acquisition, price of the acquisition, and the like. Electronic health information transaction system 120 may utilize an API to communicate with third party systems 150 to evaluate these factors which are utilized to build the content which is processed to generate the acquisition trending model.

Electronic health information transaction application processor 122 may utilize the acquisition trending model to generate a comprehensive individualized electronic health information monetization platform, that automatically determines and recommends items that are compatible with the data acquisition.

Electronic health information transaction system 120 may include an authentication processor 124 connected to electronic health information transaction application processor 122 to generate and process authentication data associated with a user trying to access the system generated health information monetization platform. Authentication processor 124 may receive user data and user input associated with an authentication request, sent from user device 140 via communication interface 142 to authenticate the user. Authentication processor 124 may evaluate the user data and user input, and upon authentication, online registry system may transmit a secure link to user device 140 that provides access to the generated health information monetization platform.

An electronic health information transaction system 120 may receive an acquisition request approval from transaction server 110, which may be configured to provide real-time data acquisition determinations. For example, electronic health information transaction system 120 may receive a notification from an acquisition system, tied to acquisition request and acceptance including at least customer identification information, customer financial information, one or more customer acquisition preferences, customer age, customer gender, customer occupation, date of acquisition, location of acquisition, purchase price for acquisition, and the like. Electronic health information transaction system 120 may have differentiated access to the acquisition server 110, other third party systems 150, which may include electronic health information systems, merchant systems, social networking systems, and the like via private Application Programming Interfaces (APIs). Electronic health information transaction system 120 may make calls to the APIs utilizing a token to provide a secure communication channel. The set of APIs may also provide a secure communication channel between a user device 140, and electronic health information transaction system 120.

Electronic health information transaction application processor 122 may retrieve information from various disparate systems, which may include third party electronic health information systems 150, and may cluster the received customer identification information, customer financial information, customer acquisition preferences, and the like to generate an acquisition trending model, which may be stored in data storage.

Electronic health information transaction application processor 122 may transmit via communication interface 128 a push notification to user device 140, where the user device. The push notification may include data indicative of the generated electronic health information monetization platform.

As depicted in FIG. 1, system 100 may include user device 140, which may be any device capable of communicating via communication interface 142, for example, Bluetooth technology, NFC technology, WiFi Direct technology, and/or the like and execute various functions to transmit and receive personal health information (which may include patient diagnoses, medications, details of acute, chronic and terminal illness, medical history, family medical history, laboratory test results, imaging records, and combinations thereof. For example, user device 140 could be an iPhone, iPod, iPad, and/or Apple Watch from Apple® or any other mobile device running Apple's iOS operating system, any device running Google's Android® operating system, including, for example, smartphones running the Android® operating system and other wearable mobile devices, such as Google Glass or Samsung Galaxy Gear Smartwatch, any device running Microsoft's Windows® Mobile operating system, and/or any other smartphone or like device.

User device 140 may also include various software components to facilitate account and payment operations including an application processor (not shown in FIG. 1). The application processor may enable execution of software applications on user device 140, which may include electronic health information transaction application 144, which may include various user interfaces, which may leverage user data, which may include user health information, wireless data connection, over-the-air data connection, or other means of data transmission to allow a user to securely access and upload data and transmit the uploaded data to transaction server 110 for purchase via electronic health information transaction system 120. The data used in the application may be transmitted, for example, from external data sources. The application and user interface may leverage information from electronic health records, genetic data, and data from health tracking devices.

Software applications on user device 140, including, for example, electronic health information transaction application 144, which may be integrated with or separate from a mobile wallet application, which may be utilized to seamlessly sell and receive payment for data by transaction server 110.

User device 140 may include for example, an input/output device (not shown in FIG. 1) and a mobile application, which may include electronic health information transaction application 144. An input/output device may include, for example, a Bluetooth device or chipset with a Bluetooth transceiver, a chip, and an antenna. The transceiver may transmit and receive information via the antenna and an interface. The chip may include a microprocessor that stores and processes information specific to a user device and provides device control functionality. Device control functionality may include connection creation, frequency-hopping sequence selection and timing, power control, security control, polling, packet processing, and the like. The device control functionality and other Bluetooth-related functionality may be supported using a Bluetooth API provided by the platform associated with the user device 140 (e.g., The Android platform, the iOS platform). Using a Bluetooth API, an application stored on a user device 140 (e.g., an electronic health record application, a patient portal application, etc.) or the device may be able to scan for other Bluetooth devices, query the local Bluetooth adapter for paired Bluetooth devices, establish RFCOMM channels, connect to other devices through service discovery, transfer data to and from other devices, and manage multiple connections. A Bluetooth API used in the methods, systems, and devices described herein may include an API for Bluetooth Low Energy (BLE) to provide significantly lower power consumption and allow a user device 140 to communicate with BLE devices that have low power requirements.

An Input/output device may include for example, I/O devices, which may be configured to provide input and/or output to user device 140 (e.g., keyboard, mouse, display, speakers, printers, modems, network cards, etc.). An input/output device also may include antennas, network interfaces that may provide or enable wireless and/or wire line digital and/or analog interface to one or more networks, such as network 130, over one or more network connections, a power source that provides an appropriate alternating current (AC) or direct current (DC) to power one or more components of user device 140, and a bus that allows communication among the various components of user device 140. An Input/output device may include a display, which may include for example output devices, such as a printer, display screen (e.g., monitor, television, and the like), speakers, projector, and the like. Although not shown, each user device 140 may include one or more encoders and/or decoders, one or more interleavers, one or more circular buffers, one or more multiplexers and/or de-multiplexers, one or more permuters and/or depermuters, one or more encryption and/or decryption units, one or more modulation and/or demodulation units, one or more arithmetic logic units and/or their constituent parts, and the like.

An input/output device may also include an NFC antenna and secure element (SE). The SE may be a hardware chip specially designed to be tamper proof. In one embodiment, the SE may be used for digitally and physically secure storage of sensitive data, including transaction card data, payment data, health records, car key identifiers, etc. The SE may, for example, store information related to a person, customer, financial institution, or other entity. The SE may store information related to personal health information (PHI). The SE may include a computer processor or other computational hardware or software. A secure element may take the form of a universal integrated circuit card (UICC) and/or a microSD card. A UICC may identify a user to a wireless operator, store contacts, enable secure connections, and add new applications and services, such as an electronic health record system.

A personal electronic health record may be associated with an authenticator associated with an individual which may allow the individual to access the health information from disparate sources, such that a remote server apparatus may be configured to issue or receive the authenticator and authorize access to the personal health electronic record by a healthcare provider server apparatus.

The authenticator may be obtained and/or transferred using a computer-readable indicia, such as, for example, a QR code, NFC token, or RFID, that can be read or otherwise transferred from the patient (e.g., via a client or patient mobile device) to a healthcare provider system. In one embodiment, the authenticator may comprise an encrypted token adapted to be passed from a patient electronic device to the healthcare provider server system, and from the health-care provider server system to the remote server apparatus to access the EHR. The patient electronic device may disconnect from the healthcare provider authentication device and/or server apparatus upon passing the authenticator, so that data from or to the personal EHR data may automatically transfer from or to the health-care provider server apparatus via the remote server apparatus and not through the patient electronic device.

An input/output device may enable Industry Standard NFC Payment Transmission. For example, the input/output device may enable two loop antennas to form an air-core transformer when placed near one another by using magnetic induction. The input/output device may operate at 13.56 MHz or any other acceptable frequency. Also, the input/output device may provide for a passive communication mode, where the initiator device provides a carrier field, permitting answers by the target device via modulation of existing fields. Additionally, the input/output device also may provide for an active communication mode by allowing alternate field generation by the initiator and target devices.

The input/output device may deactivate the RF field while awaiting data. The attachment may use Miller-type coding with varying modulations, including 100% modulation. The attachment may also use Manchester coding with varying modulations, including a modulation ratio of 10%. Additionally, the attachment may be capable of receiving and transmitting data at the same time, as well as checking for potential collisions when the transmitted signal and received signal frequencies differ.

The input/output device may be capable of utilizing standardized transmission protocols, for example but not by way of limitation, ISO/IEC 14443 A/B, ISO/IEC 18092, MiFare, FeliCa, tag/smartcard emulation, and the like. Also, the input/output device may be able to utilize transmission protocols and methods that are developed in the future using other frequencies or modes of transmission. The input/output device may also be backwards-compatible with existing techniques, for example RFID. Also, the system may support transmission requirements to meet new and evolving standards including internet based transmission triggered by NFC.

The current location of user device 140 may be determined using many different technologies such as GPS technology, Internet-based technology, etc., which may utilize location data. By way of example, location data may include, but is not limited to GPS data, assisted GPS data, IP address data, cell ID data, received signal strength indication (RSSI) data, wireless fingerprinting data, inertial sensor data (e.g., compass or magnetometer data, accelerometer data, and/or gyroscope data), barometer data, ultrasonic data (e.g., radio-frequency identification (RFID) data, near-field communication (NFC) data), Bluetooth data, and/or terrestrial transmitter data.

User device 140 may also include various software components to facilitate the retrieval and secure transmission of health information including an App Processor. For example, mobile device 110 may include an operating system such as, for example, the iOS operating system from Apple, the Google Android operating system, and the Windows Mobile operating system from Microsoft. User device 140 may also include, without limitation, software applications such as electronic health record and patient portal systems, electronic health information transaction applications 144, an NFC application programming interface, and software to enable touch sensitive displays. Mobile device manufacturers may provide software stacks or Application Programming Interfaces (APIs) which allow software applications to be written on top of the software stacks. For example, mobile device manufacturers may provide, without limitation, a card emulation API to enable NFC card emulation mode, a logic link control protocol (LLCP) API for peer-to-peer communication between mobile devices, a Bluetooth API supporting BLE, and a real-time data (RTD) API and a NFC Data Exchange Format (NDEF) API for reading/writing.

The App Processor may enable execution of software applications on user device 140 In various embodiments, the App Processor may cooperate with the NFC technology.

The App Processor may enable execution of a monetization platform application, which may include various user interfaces, which may leverage health data, wireless data connection, over-the-air data connection, or other means of data transmission. The data used in the application may be transmitted, for example, from external data sources.

Software applications on user device 140 may include, for example, electronic health information transaction application 144, which may be integrated with or separate other applications, which may be utilized to generate a comprehensive individualized electronic health information monetization platform.

Data storage 146 may store data and/or components to enable the generation, transmission and processing of user data, which may include data received from third party electronic health information systems 150, which may include EHR systems, DNA Genome systems, which provide genetic, health and ancestry data, and health tracking devices, which may include wearables such as a Fitbit, Nike fitness tracker, Samsung fitness tracker, Microsoft Band, and/or Apple Watch.

System 100 may also communicate with social networking sites, which may include, without limitation, Facebook, MySpace, Google+, LinkedIn, Twitter, Pinterest, Instagram, etc. The social networking site may include a plurality of social networking accounts created by one or more users. Electronic Health Information Transaction System 120 may establish a secure connection with social networking systems, and may utilize a secure communication interface to share a secure link that provides access to a user's individualized electronic health information monetization platform and associated marketplace.

Referring to FIG. 2, system 200 may include a system and device to generate a comprehensive individualized electronic health information monetization platform. For example, system 200 may include client device 202, which may be similar to user device 110, a network 204, which may be similar to network 130, a front-end controlled domain 206, a back-end controlled domain 212, and a backend system 218. Front-end controlled domain 206 may include one or more load balancers 208 and one or more web servers 210. Back-end controlled domain 212 may include one or more load balancers 214 and one or more application servers 216.

User device 202 may be a network-enabled computer. As referred to herein, a network-enabled computer may include, but is not limited to: e.g., any computer device, or communications device including, e.g., a server, a network appliance, a personal computer (PC), a workstation, a mobile device, a phone, a handheld PC, a personal digital assistant (PDA), a thin client, a fat client, an Internet browser, or other device. The one or more network-enabled computers of the example system 200 may execute one or more software applications to enable, for example, network communications.

User device 202 may include an iPhone, iPod, iPad, and/or Apple Watch from Apple® or any other mobile device running Apple's iOS operating system, any device running Google's Android® operating system, including for example, Google's wearable device, Google Glass, any device running Microsoft's Windows® Mobile operating system, and/or any other smartphone or like wearable mobile device.

Network 204 may be one or more of a wireless network, a wired network, or any combination of a wireless network and a wired network. For example, network 204 may include one or more of a fiber optics network, a passive optical network, a cable network, an Internet network, a satellite network, a wireless LAN, a Global System for Mobile Communication (GSM), a Personal Communication Service (PCS), a Personal Area Networks, (PAN), D-AMPS, Wi-Fi, Fixed Wireless Data, IEEE 802.11b, 802.15.1, 802.11n, and 802.11g or any other wired or wireless network for transmitting and receiving a data signal.

In addition, network 204 may include, without limitation, telephone lines, fiber optics, IEEE Ethernet 902.3, a wide area network (WAN), a local area network (LAN) or a global network such as the Internet. Also, network 204 may support an Internet network, a wireless communication network, a cellular network, or the like, or any combination thereof. Network 204 may further include one network, or any number of example types of networks mentioned above, operating as a stand-alone network or in cooperation with each other. Network 204 may utilize one or more protocols of one or more network elements to which they are communicatively couples. Network 204 may translate to or from other protocols to one or more protocols of network devices. Although network 204 is depicted as a single network, it should be appreciated that according to one or more embodiments, network 204 may comprise a plurality of interconnected networks, such as, for example, the Internet, a service provider's network, a cable television network, corporate networks, and home networks.

Front-end controlled domain 206 may be implemented to provide security for backend 218. Load balancer(s) 208 may distribute workloads across multiple computing resources, such as, for example computers, a computer cluster, network links, central processing units or disk drives. In various embodiments, load balancer(s) 210 may distribute workloads across, for example, web server(s) 216 and/or backend 218 systems. Load balancing aims to optimize resource use, maximize throughput, minimize response time, and avoid overload of any one of the resources. Using multiple components with load balancing instead of a single component may increase reliability through redundancy. Load balancing is usually provided by dedicated software or hardware, such as a multilayer switch or a Domain Name System (DNS) server process.

Load balancer(s) 208 may include software that monitoring the port where external clients, such as, for example, user device 202, connect to access various services of a financial institution, for example. Load balancer(s) 208 may forward requests to one of the application servers 216 and/or backend 218 servers, which may then reply to load balancer 208. This may allow load balancer(s) 208 to reply to user device 202 without user device 202 ever knowing about the internal separation of functions. It also may prevent mobile devices from contacting backend servers directly, which may have security benefits by hiding the structure of the internal network and preventing attacks on backend 218 or unrelated services running on other ports, for example.

A variety of scheduling algorithms may be used by load balancer(s) 208 to determine which backend server to send a request to. Simple algorithms may include, for example, random choice or round robin. Load balancers 208 also may account for additional factors, such as a server's reported load, recent response times, up/down status (determined by a monitoring poll of some kind), number of active connections, geographic location, capabilities, or how much traffic it has recently been assigned.

Load balancers 208 may be implemented in hardware and/or software. Load balancer(s) 208 may implement numerous features, including, without limitation: asymmetric loading; Priority activation: SSL Offload and Acceleration; Distributed Denial of Service (DDoS) attack protection; HTTP/HTTPS compression; TCP offloading; TCP buffering; direct server return; health checking; HTTP/HTTPS caching; content filtering; HTTP/HTTPS security; priority queuing; rate shaping; content-aware switching; client authentication; programmatic traffic manipulation; firewall; intrusion prevention systems.

Web server(s) 210 may include hardware (e.g., one or more computers) and/or software (e.g., one or more applications) that deliver web content that can be accessed by, for example a client device (e.g., user device 202) through a network (e.g., network 204), such as the Internet. In various examples, web servers, may deliver web pages, relating to, for example, online patient portal systems and the like, to clients (e.g., user device 202). Web server(s) 210 may use, for example, a hypertext transfer protocol (HTTP/HTTPS or sHTTP) to communicate with user device 202. The web pages delivered to user device may include, for example, HTML documents, which may include images, style sheets and scripts in addition to text content.

A user agent, such as, for example, a web browser, web crawler, or native mobile application, may initiate communication by making a request for a specific resource using HTTP/HTTPS and web server 210 may respond with the content of that resource or an error message if unable to do so. The resource may be, for example a file on stored on backend 218. Web server(s) 210 also may enable or facilitate receiving content from user device 202 so user device 202 may be able to, for example, submit web forms, including uploading of files.

Web server(s) also may support server-side scripting using, for example, Active Server Pages (ASP), PHP, or other scripting languages. Accordingly, the behavior of web server(s) 210 can be scripted in separate files, while the actual server software remains unchanged.

Load balancers 214 may be similar to load balancers 208 as described above.

Application server(s) 216 may include hardware and/or software that is dedicated to the efficient execution of procedures (e.g., programs, routines, scripts) for supporting its applied applications. Application server(s) 216 may comprise one or more application server frameworks, including, for example, Java application servers (e.g., Java platform, Enterprise Edition (Java EE), the .NET framework from Microsoft®, PHP application servers, and the like). The various application server frameworks may contain a comprehensive service layer model. Also, application server(s) 216 may act as a set of components accessible to, for example, a financial institution, or other entity implementing system, through an API defined by the platform itself. For Web applications, these components may be performed in, for example, the same running environment as web server(s) 210, and application servers 216 may support the construction of dynamic pages. Application server(s) 216 also may implement services, such as, for example, clustering, fail-over, and load-balancing. In various embodiments, where application server(s) 216 are Java application servers, the web server(s) 216 may behaves like an extended virtual machine for running applications, transparently handling connections to databases associated with backend 218 on one side, and, connections to the Web client (e.g., user device 202) on the other.

Backend 218 may include hardware and/or software that enables the backend services of, for example, a financial institution, merchant, or other entity that maintains a distributed system similar to system 200. For example, backend 218 may include, a system of audit applications, encryption applications, BLE/Bluetooth connection platforms, a rewards platform, one or more platforms that provide mobile services, one or more platforms that provide online services, and/or a location system, which may include additional capabilities, such as health data generation, processing, and/or transmission. Backend 218 may be associated with various databases, including account databases that maintain, for example, patient health information (e.g., demographic data, medical historical data, and the like), patient financial data; patient insurance data, health care provider data, connection information (e.g., public/private key pairs, UUIDs, device identifiers, and the like) and the like. Backend 218 also may be associated with one or more servers that enable the various services provided by system 200.

The system may assimilate data from disparate (heterogeneous) sources that is able to function regardless of the disparate data protocols of the associated data sources. The health data may be retrieved and/or generated from various sources. Examples of such sources may include computer systems used in clinical trials (e.g., electronic data capture (EDC), safety, or randomization systems), computer systems used by healthcare professionals (e.g., electronic health records (EHR) or electronic medical records (EMR) systems), computer systems used by consumers (e.g., electronic patient-reported outcomes (ePRO) systems), medical devices (e.g., a blood glucose device used in a home, or an ECG in a clinic), consumer devices (e.g., a blood pressure cuff used on a phone, or an activity tracker), and centralized systems for storage of data from devices (e.g., in the cases of activity trackers, which often send their data via the Internet to the “cloud”). Other devices capturing health data may be utilized as well, including data associated with Internet of Things (“IoT”), such as Vital Connect and Actigraph applications from different clients. (The Internet of Things interconnects embedded devices on the Internet.).

The system may seamlessly process data from disparate sources to generate a comprehensive individualized record that may be utilized to generate an individualized electronic health information monetization platform. Typically a health-care provider (HCP) maintains a unique patient database organized frequently using a unique EMR software. Accordingly, the data are fragmented between different HCPs and are never consolidated, neither physically nor logically, in a single data structure. Additionally, patients have information about lifestyle and life choices that is not recorded. More so, other HCPs, such as, but not limited to, dentists, optometrists, psychologists, nutritionists, physical therapists, and clinicians providing basic medical care in pharmacies, have relevant data but are largely likewise ignored by HCPs

The systems may provide or export data in different formats or via specific types of interchange protocols or standards, such as HL7 (Health Level Seven), CDISC/ODM, and E2B. In the United States, HL7's messaging standard is supported by most major medical information systems vendors. CDISC standards, set by the standards developing organization (SDO) of the same name, are platform-independent data standards used in clinical research. Even with the use of communication standards, the data utilized by the system may not be standardized.

The system may utilize a master data source generator that may take as inputs data from disparate sources, e.g., data from public databases, data from private databases, and data from users. Such data may differ in data type, data structure, as well as native naming conventions and accessibility. The system may utilize a data cleanser, which may utilize bioinformatics, artificial intelligence and machine learning to clean the data such that data differing in data type and data structure may be synthesized. Additionally, the system may also automatically de-identify data so that it is indecipherable from the standpoint of the ability to trace a particular data item to a specific subject.

As such, the system may provide interoperability with electronic medical records (EMR), payer (e.g., insurance, third-party payer) databases, and government agencies (e.g., Food and Drug Administration, Medicaid, Medicare), and aids in integration with emerging scientific areas and technologies, and the monetization of such personal health information. Although described in some embodiments in terms of electronic health record systems, the invention benefits industries other than those in other domains that include entities and data from disparate, heterogeneous sources.

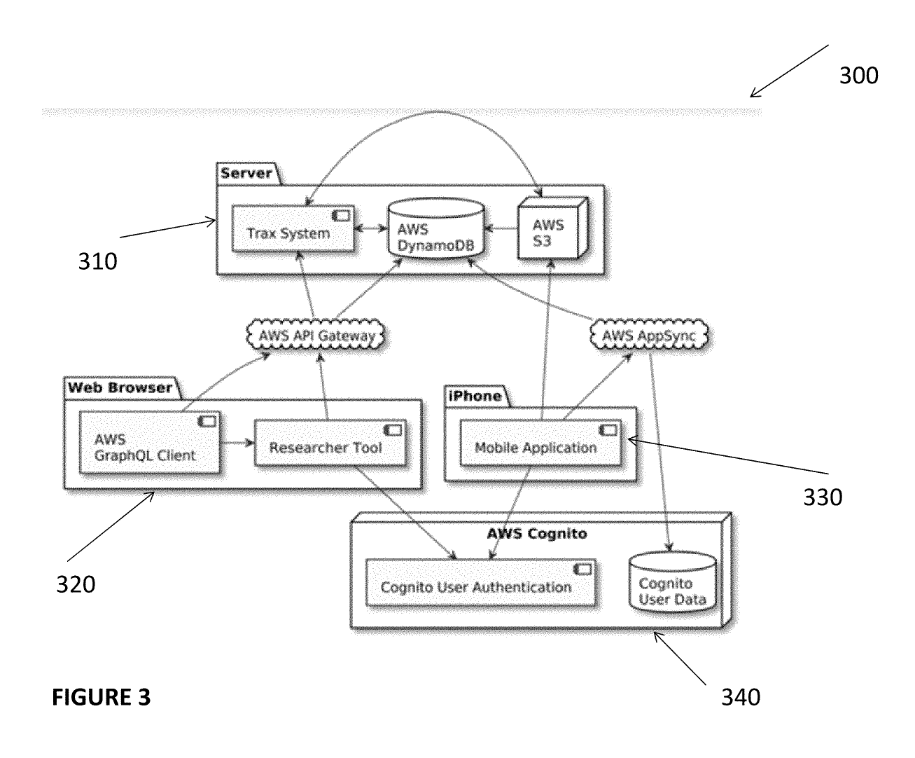

FIG. 3 depicts an example communication architecture 300 for generating a comprehensive individualized electronic health information monetization platform. A researcher tool web application 320 may communicate with electronic health information transaction system 310 via a data query and communication protocol, which may include GraphQL client for synchronization of user account information, user preferences and settings. The system may generate a schema, which may include a GraphQL schema for all data being set to or retrieved from the electronic health information transaction system. The system may dynamically generate database storage based on the associated schema.

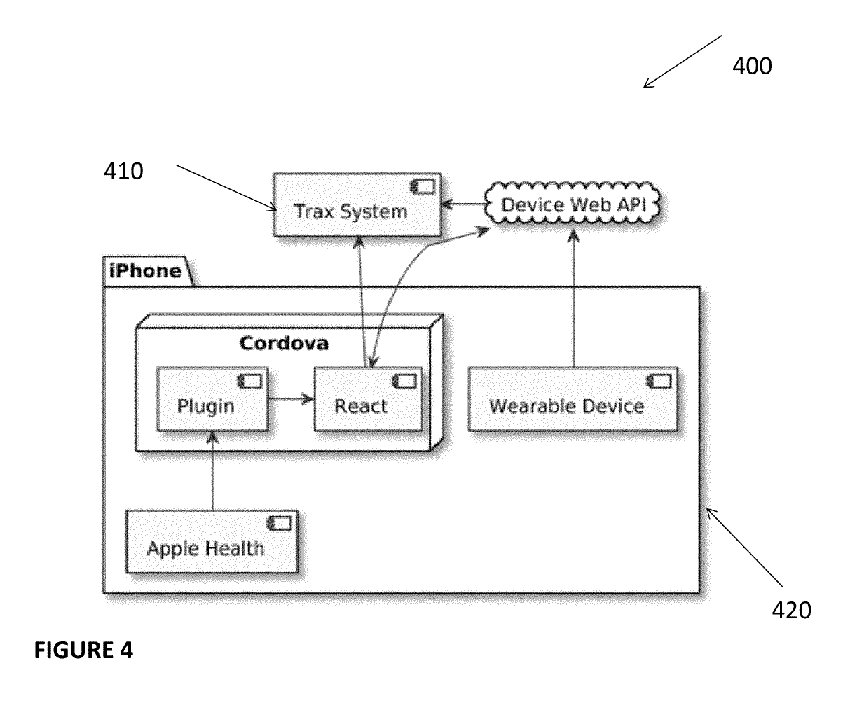

FIG. 4 depicts an example data access architecture 400 for generating a comprehensive individualized electronic health information monetization platform. Mobile application platform 420 may use a plugin to access a third party electronic health information system's data. For example, the platform may utilize Apple Health's data to access EHR data. The EHR data may be delivered in a particular format, for example JSON format, structured according to the FHIR (Fast Healthcare Interoperability Resources) specification. As soon as a user connects to the associated data source, the mobile application may upload encrypted data to the electronic health information transaction system. For example the data may be encrypted using HTTPS. The data may be stored in data storage on the electronic health information transaction system, which may include file storage, and the stored data may be linked to a user's account. For example, the data may be linked via a unique identifier. The data may be encrypted utilizing keys, for example AWS KMS-Managed Keys (SSE-KMS).

For each data source supported by the mobile application, the application may need to implement an API for that data source, a device web API. For example, to support downloading data from a wearable device, the mobile application will need to implement the associated wearable API. An API that requires a server-side connection may also utilize a REST utility function.

As each data source file is uploaded, the electronic health information transaction system 410 may parse the data and build a search index. The data may be parsed such that it may be used by the Research Tool search filters to facilitate a data acquisition transaction. The index data may be stored in a data source, which may include a database. The system may generate a data density for the associated date, which may include low, medium, or high.

Data associated with a research study may be stored in a folder in the file storage system, with one file for storing EHR data, one for wearable device, and one for genomic data. Data may be appended to these files as the studies run and data is gathered. A temporary download link may be generated using the file system API to download the files.

An authentication processor may provide secure user authentication, which may facilitate login via email address, two-factor authentication via a transmitted security, which, for example, may be transmitted via SMS, and user authentication via third party applications. The authentication processor may store user settings for the mobile application in authentication data storage.

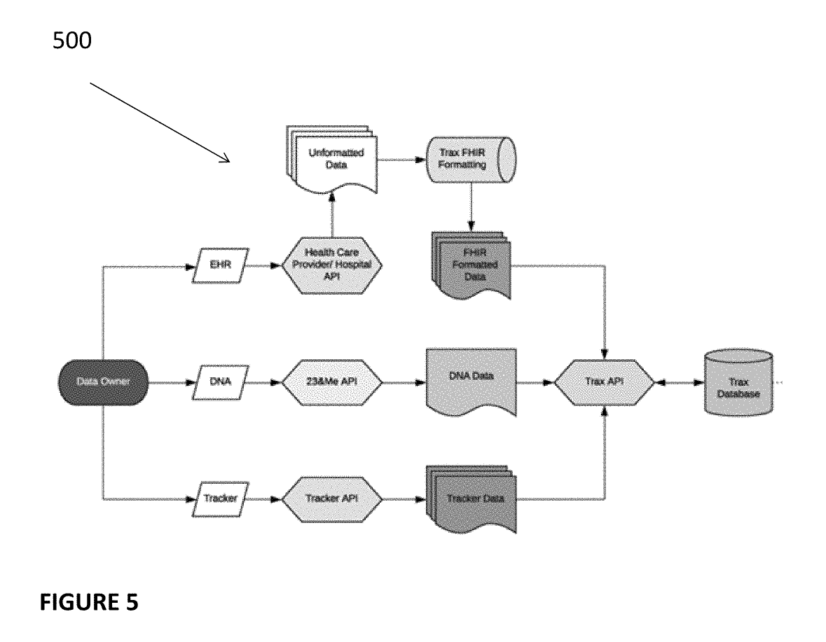

FIG. 5 provides a diagram of data input processing for a comprehensive individualized health information monetization platform.

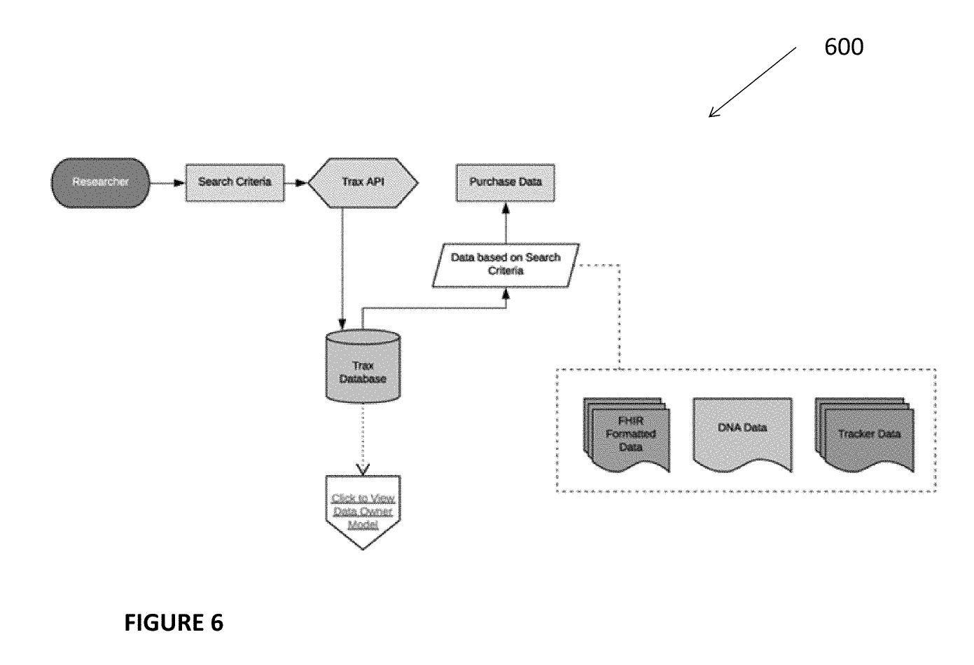

FIG. 6 provides a diagram of data query and retrieval processing for a comprehensive individualized health information monetization platform.

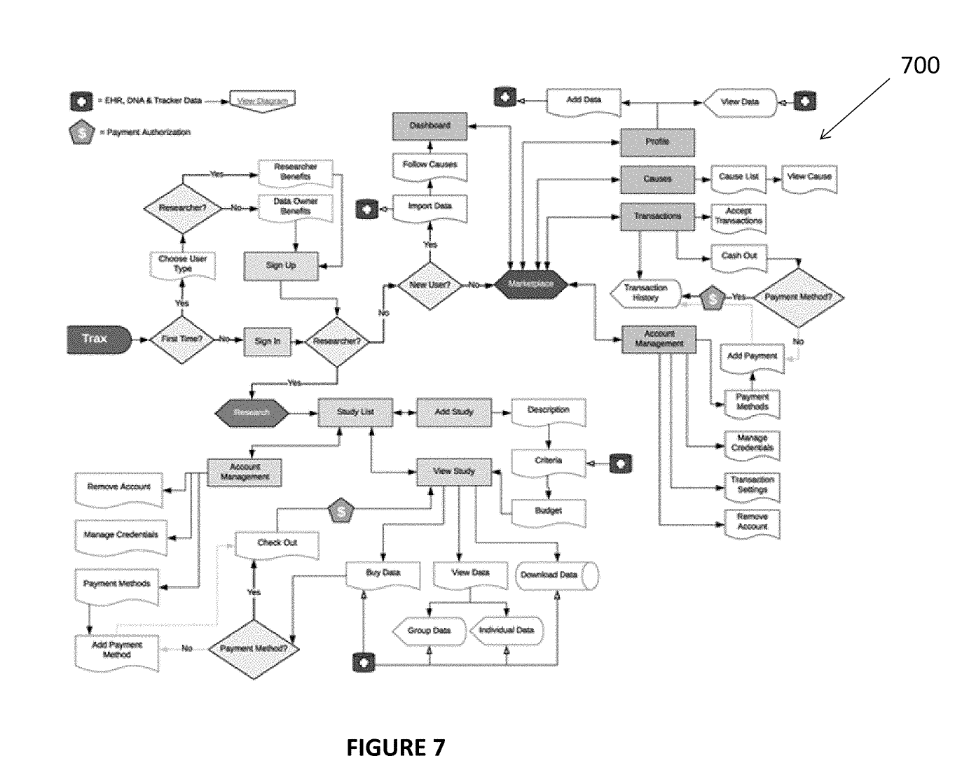

FIG. 7 provides a flowchart for a method for generating a comprehensive individualized electronic health information platform, which may include research, account management, payment, user profile, data importation, data downloading, and dashboard generation processing.

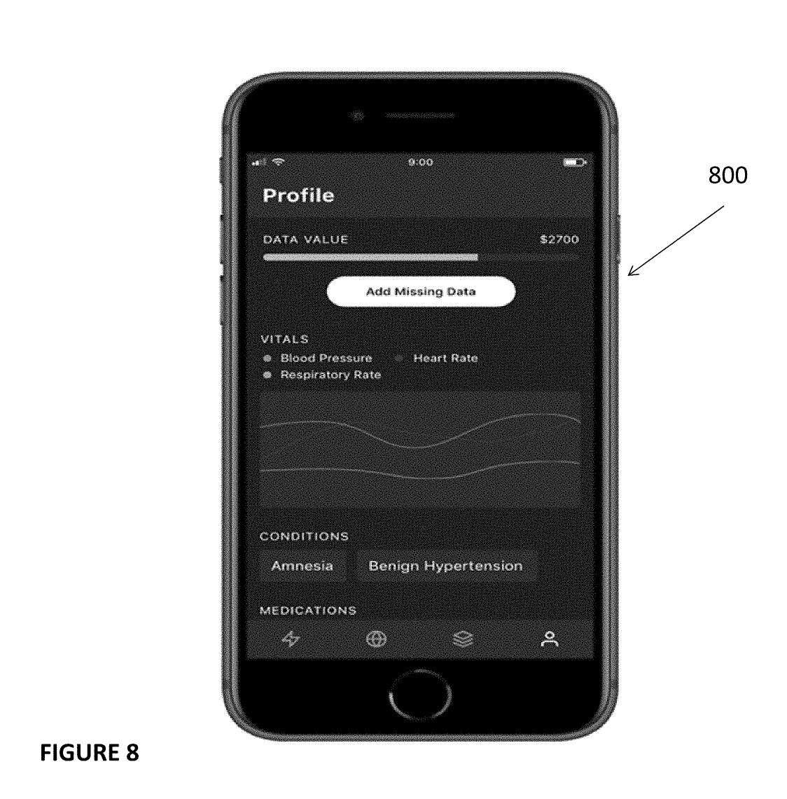

FIGS. 8-12 provide example customizable interfaces for a comprehensive individualized electronic health information platform.

Utilizing such customizable interfaces, a user may log in to a mobile application. The user may be authenticated utilizing multi factor authentication. For example, the system may generate a code that may be sent to mobile device associated with account and/or an email address associated with the account, and the user may be required to enter the code to log on. Similarly, such codes may also be generated and required to be input for transaction processing, for example when a user approves a transaction for associated user data to be purchased by a data acquisition system, for example, by a researcher. The mobile application may authenticate to the system by evaluating associated mobile application user credentials. A user may also utilize single sign-on (SSO) to log on to the system utilizing user credentials for third party systems, which may include web, email, and social media credentials.

The system may prompt a user to import data. The mobile application may communicate over secure encrypted protocols with the system, and data may be automatically loaded to the health information transaction system upon connection with the data sources upon authorization from the user to upload data to a system server. The data sources may include electronic health information data from EHR systems DNA Genome health and ancestry data (for example data from a 23andMe kit), and data from health tracking devices, which may include supported health and fitness tracking applications and devices. For each specific data source, a user may specify which particular the user authorizes to be uploaded to the system for example, basal body temperature, body temperature, cervical mucous quality, menstruation, ovulation test result, sexual activity, spotting, weight, blood pressure, imaging, and/or lab results, for example. The Data may be dynamically updated and uploaded in real time as is updated in third party systems.

The system may parse and index the imported data to generate an associated data density, which may include a data density of low, medium, or high. For example, the data density score for a user who has uploaded data from multiple sources and has agreed to share more particular data elements will have a higher data density score, as an increase in the number of data sources and associated data elements increase data density.

The mobile application may provide a dashboard interface, which may provide suggested data acquisition parties to follow, for example, researchers from the transaction. The system may generate dynamic smart suggestions based on a user's health data.

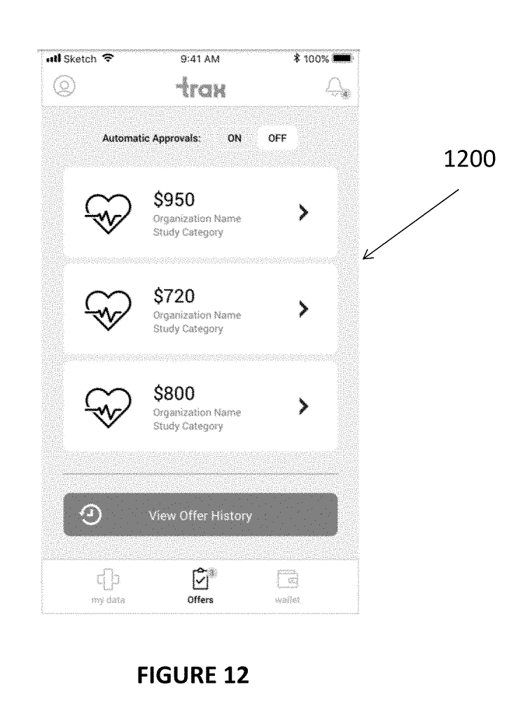

The system may also generate and display a value score and associated data net worth based on a user's health data and a likelihood of being purchased as compared to other users (for example, 40% better chance of being purchased). This system generated data net worth may be dynamically updated in real time, as the data acquisition system purchases data and users upload additional data, and is based on average amount paid to other users with data similar to yours, and on the number and type of data sources and data density of each source). The system may generated a prediction for a time of when the data may be requested to be purchased and an estimated cost value of the data, which may be displayed to the user via the mobile application. Additionally, the user may be prompted to add more data, all illustrated in FIG. 8. The system display real time updates to the data. A user may receive an alert on a mobile device, which may be via a push notification even if the user is not logged into the mobile application, such that the user receive the alert that an entity, such as a researcher, wants to purchase a package of the users data. For each data purchase offer, a user may be presented with information regarding the purchaser, which may include research study information. A setting stored in a user profile of the application may specify whether the user must affirmatively review each purchase offer, or whether all or particular data purchase offers are automatically approved. A log of a user's offers may be stored including those accepted by the user and those declined, as illustrated by FIG. 12. The system may also generate an alert that is transmitted to the user via the mobile application that the user's data would be valued more if additional data were added, and may provide the option to add such data.

The mobile application interface may provide a transaction tab that may display financial transaction information based on the purchase of data, which may include a history of transaction data. As data is purchased via a transaction server, funds may be added to a user's electronic mobile wallet, which may store the money received from accepted offers. The mobile application is connected to financial institution and account providers systems, and as such, a user may cash out to receive funds including, for example, via a credit, or via money deposited into a user's account at a bank and/or other financial institution or third party system.

A web application framework may be utilized by data acquisition entities to gain access to large amounts of data. For example, a researcher tool may be utilized to gain access to large amounts of health data included associated EHR, DNA, and tracker data of system users.

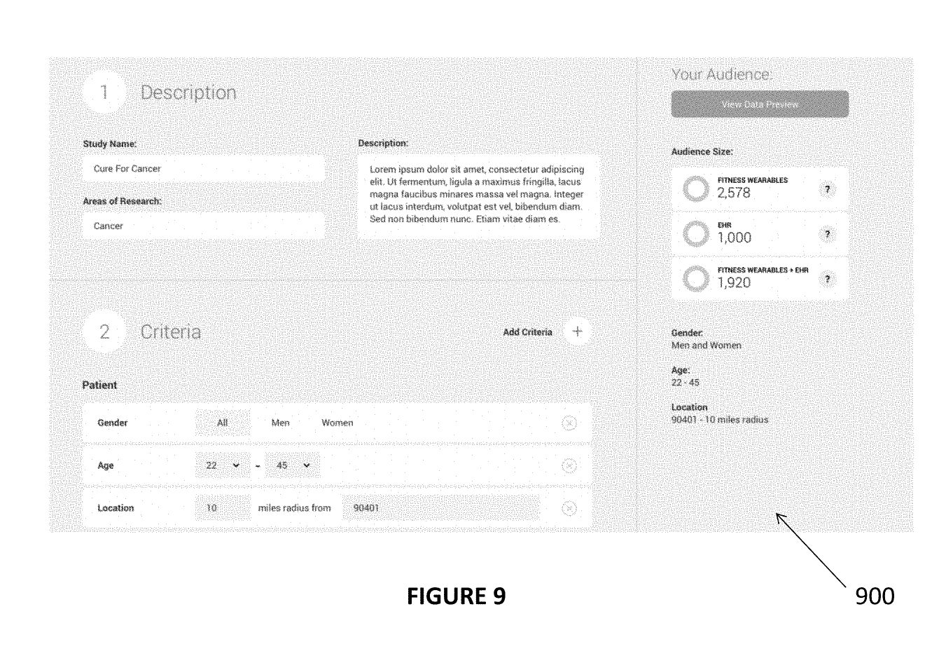

For example, in one embodiment, a researcher log in to data acquisition application, which may include a web interface or app on a mobile device, to purchase data. As depicted in FIG. 9, a researcher may add a study, and may designate criteria for a study which corresponds to data the researcher may seek to purchase. For example, for a studying regarding a cure for cancer, a researcher may specify criteria is to only analyze men, of a particular age, from a particular location. An electronic health information processor may evaluate potential data to be purchased to establish proof of authenticity of the data, which may be established by information, for example, from a user's mobile device that was utilized to upload the data, and certification of the data source. The system generated criteria may select patients from at least one of the following: gender, age, location, allergies, prescriptions, observations, conditions, procedures, immunizations, dispenses, family history. The researcher tool interface may dynamically display how certain criteria, and filtering patient samples based on the criteria affects the number of available samples.

The system may automatically send alerts when additional patient data is available that meets certain criteria. The researcher tool may be utilized to set a budget for purchasing data. The budget may be generated and based on the selection of high density data, versus medium or low density data for a defined sample size.

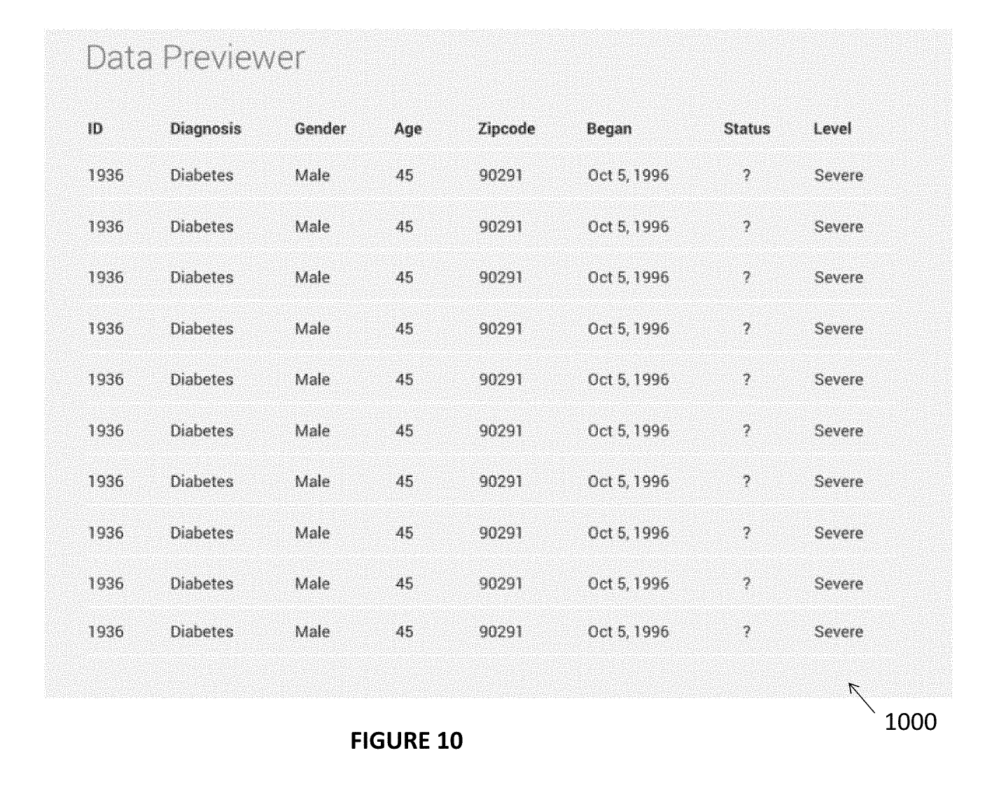

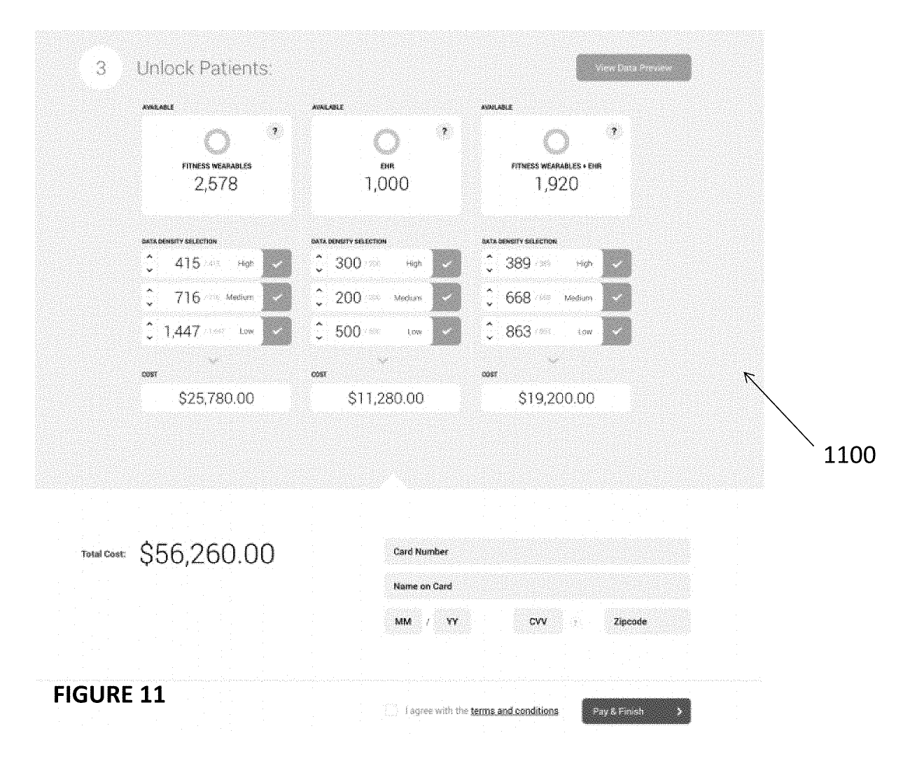

As illustrated in FIG. 10, the system may display number of patient users with specific data sources: EHR; Trackers and EHR; Genome and EHR; and Genome, Trackers, EHR corresponding to the total number of patient samples available that meet the selected criteria. As illustrated in FIG. 11, a system generated data summary of available sample data may include counts and pricing for each data group and each data density level. Unlocking and previewing of patient data may involve reserving funds, for example placing a temporary hold, from a researchers account. The interface may provide a payment interface and may display the cost of the associated patient data samples. The system may be utilized to export purchased data as it's collected in real time, locally to the researcher server and/or to other third party systems, in real time once the data purchase is approved.

The present disclosure is not to be limited in terms of the particular embodiments described in this application, which are intended as illustrations of various aspects. Many modifications and variations can be made without departing from its spirit and scope, as may be apparent. Functionally equivalent methods and apparatuses within the scope of the disclosure, in addition to those enumerated herein, may be apparent from the foregoing representative descriptions. Such modifications and variations are intended to fall within the scope of the appended representative claims. The present disclosure is to be limited only by the terms of the appended representative claims, along with the full scope of equivalents to which such representative claims are entitled. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It may be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It may be further understood by those within the art that if a specific number of an introduced claim recitation is intended, such an intent may be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It may be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” may be understood to include the possibilities of “A” or “B” or “A and B.”

The foregoing description, along with its associated embodiments, has been presented for purposes of illustration only. It is not exhaustive and does not limit the invention to the precise form disclosed. Those skilled in the art may appreciate from the foregoing description that modifications and variations are possible in light of the above teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Accordingly, the invention is not limited to the above-described embodiments, but instead is defined by the appended claims in light of their full scope of equivalents.

In the preceding specification, various preferred embodiments have been described with references to the accompanying drawings. It may, however, be evident that various modifications and changes may be made thereto, and additional embodiments may be implemented, without departing from the broader scope of the invention as set forth in the claims that follow. The specification and drawings are accordingly to be regarded as an illustrative rather than restrictive sense.

1. A system for generating a comprehensive individualized electronic health information monetization platform, comprising:
a data storage containing user identification information, user electronic health information, user financial information, and one or more user acquisition preferences;
a data source generator that:
takes as inputs user electronic health information from third party electronic health information systems;
clusters the received user identification information, user electronic health information, user financial information, and one or more user acquisition preferences
via a data cleanser, utilizes bioinformatics, artificial intelligence and machine learning technologies to clean and synthesize the received user electronic health information based on data type and structure;
de-identifies the synthesized data; and
encrypts the de-identified synthesized data for transmission; and
an electronic health information transaction processor that:
uploads the de-identified synthesized data from the data source generator;
parses the de-identified synthesized data to build a search index;
evaluates the data to generate a data acquisition trending model;
generates a comprehensive individualized electronic health information monetization platform utilizing the data acquisition trending model that determines and recommends data samples that are compatible; and
transmits via a communication interface, a push notification to a user device, wherein the push notification includes a data acquisition request received from transaction server;
wherein the data storage stores the generated data acquisition trending model.

2. The system of claim 1, further comprising an authentication processor connected to the electronic health information transaction processor that receives user data and user input associated with an authentication request, sent from the user device via the communication interface, to authenticate the user, wherein upon authentication of the user based on evaluation of the user data and user input, the system transmits a secure link to the user device that provides access to the comprehensive individualized electronic health information monetization platform.

3. The system of claim 2, wherein the authentication processor confirms the location of the user device over a wireless connection by evaluating a unique user ID-secure link token pair.

4. The system of claim 2, wherein the authentication processor confirms the authenticity of the data source of the user electronic health information from third party electronic health information systems by evaluating a unique user ID-secure link token pair.

5. The system of claim 1, wherein the electronic health information transaction processor receives recommended data acquisition requests and automatically updates the comprehensive individualized electronic health information monetization platform with the received recommended data acquisition requests.

6. The system of claim 1, wherein the electronic health information transaction processor establishes a secure communication interface with a third party system configured to share a secure link that provides access to the comprehensive individualized electronic health information monetization platform.

7. The system of claim 1, wherein the electronic health information transaction processor utilizes the data acquisition trending model to generate a user's data net worth based on a system generated data density score associated with the user's electronic health information.

8. The system of claim 1, wherein the electronic health information transaction processor transmits via a communication interface, a push notification to the user device, wherein the push notification includes a secure link to input additional user to increases the user's generated data net worth.

9. The system of claim 1, wherein upon receipt of acceptance of the data acquisition request by the user device, the transaction server processes an associated transaction.
a data acquisition request received from transaction server

10. The system of claim 1, wherein the electronic health information application processor links the de-identified synthesized data with the user's account via a unique identifier.

11. A method for generating a comprehensive individualized electronic health information monetization platform, comprising:
receiving as inputs user electronic health information from third party electronic health information systems;
clustering the received user identification information, user electronic health information, user financial information, and one or more user acquisition preferences
utilizing bioinformatics, artificial intelligence and machine learning technologies to clean and synthesize the received user electronic health information based on data type and structure;
de-identifying the synthesized data;
encrypting the de-identified synthesized data for transmission;
uploading the de-identified synthesized data from the data source generator;
parsing the de-identified synthesized data to build a search index;
evaluating the data to generate a data acquisition trending model;
storing the generated data acquisition trending model in a data storage;
generating a comprehensive individualized electronic health information monetization platform utilizing the data acquisition trending model that determines and recommends data samples that are compatible; and
transmitting, via a communication interface, a push notification to a user device, wherein the push notification includes a data acquisition request received from transaction server.

12. The method of claim 11, further comprising receiving user data and user input associated with an authentication request, sent from the user device via the communication interface, to authenticate the user, wherein upon authentication of the user based on evaluation of the user data and user input, transmitting a secure link to the user device that provides access to the comprehensive individualized electronic health information monetization platform.

13. The method of claim 12, further comprising confirming the location of the user device over a wireless connection by evaluating a unique user ID-secure link token pair.

14. The method of claim 12, further comprising confirming the authenticity of the data source of the user electronic health information from third party electronic health information systems by evaluating a unique user ID-secure link token pair.

15. The method of claim 11, further comprising receiving recommended data acquisition requests and automatically updating the comprehensive individualized electronic health information monetization platform with the received recommended data acquisition requests.

16. The method of claim 11, further comprising establishing a secure communication interface with a third party system configured to share a secure link that provides access to the comprehensive individualized electronic health information monetization platform.

17. The method of claim 11, further comprising utilizing the data acquisition trending model to generate a user's data net worth based on a system generated data density score associated with the user's electronic health information.

18. The method of claim 11, further comprising transmitting via a communication interface, a push notification to the user device, wherein the push notification includes a secure link to input additional user to increases the user's generated data net worth.

19. The method of claim 11, further comprising processing an associated transaction.
a data acquisition request received from the transaction server upon receipt of acceptance of the data acquisition request by the user device.

20. The method of claim 11, further comprising linking the de-identified synthesized data with the user's account via a unique identifier.
